(19) United States
(12) Patent Application Publication
(10) Pub. No.: US 2003/0194388 A1
(43) Pub. Date: Oct. 16, 2003

(54) Process for the preparation of ether carboxylic acids with a low setting point

(75) Inventors: Uwe Dahlmann, Heidelberg (DE); Rainer Kupfer, Hattersheim (DE); Eduard Masvidal Climent, Tarragona (ES)

Correspondence Address: Clariant Corporation, Industrial Property Department, 4000 Monroe Road, Charlotte, NC, 28205, US

(73) Assignee: Clariant GmbH

(21) Appl. No.: 10/409,498

(22) Filed: Apr. 8, 2003

(30) Foreign Application Priority Data
Apr. 12, 2002 (DE) 10217208.0

(51) Int. Cl. A61K 7/075; A61K 7/08; C23G 1/00; C07C 59/01

(52) U.S. Cl. 424/70.22; 562/567; 510/245; 510/360

(57) ABSTRACT

The invention provides a process for the preparation of compounds of the formula (1)

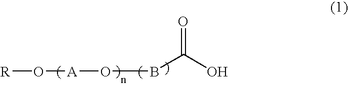

in which

A is C₂- to C₄-alkylene,

B is C₁- to C₄-alkylene

n is a number from 1 to 100, and

R is C₁- to C₃₀-alkyl, C₂- to C₃₀-alkenyl, or C₆- to C₃₀-aryl,

by alkylating a basic mixture of oxethylated alcohols of the formula

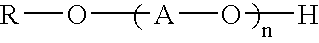

and alkoxides thereof with a C₂- to C₅-chlorocarboxylic acid, and purifying the basic intermediate obtained in this way, following acidification, by washing with aqueous sulfate solution until the ether carboxylic acid obtained in this way has a conductivity of <1000 μS/cm.

The present invention relates to a process for the preparation of ether carboxylic acids with a low setting point, and to the use thereof as metal-working auxiliaries, in cosmetic formulations and as detergents in washing compositions.

Ether carboxylic acids, i.e. organic carboxylic acids which carry one or more ether bridges in addition to the carboxyl function, or their alkali metal or amine salts, are known as mild detergents with a high lime soap dispersing power. They are used both in detergent and cosmetics formulations, but also in technical applications, such as, for example, metal-working liquids and cutting fluids.

According to the prior art, ether carboxylic acids (ECA) are prepared either by alkylation of alcohol or fatty alcohol oxethylates or oxpropylates with chloroacetic acid derivatives (Williamson ether synthesis) or from the same starting materials by oxidation with various reagents (atmospheric oxygen, hypochlorite, chlorite) under catalysis with various catalysts. The Williamson ether synthesis represents the process which is most common in industry for the preparation of ECA, primarily due to the cost-effect relationship, although products prepared by this process have serious shortcomings with regard to handlability for the user, such as, for example, solubility behavior, aggregate state at low temperatures and storage stability.

These shortcomings can essentially be attributed to secondary constituents as a consequence of the process. For example, despite the use of excesses of the corresponding chloroacetic acid derivative, only conversions of about 70-85% are achieved, meaning that residual amounts of oxethylate and fatty alcohol on which the oxethylate is based remain in the end product.

Furthermore, the excess of the chloroacetic acid derivative to be used results in by-products, such as, for example, glycolic acid, diglycolic acid and derivatives thereof, which are a significant cause of product aging and in some cases may cause problems relating to the solubility behavior.

A further disadvantage of the Williamson synthesis is the high burden placed on the reaction products by sodium chloride (content about 1%), which, in aqueous solutions, represents a significant cause of pitting corrosion.

DE-A-199 28 128 discloses a process for the preparation of ether carboxylic acids with a low residual alcohol content by firstly reacting fatty alcohols with alkylene oxides using noncatalytic amounts of alkali metal catalyst (NaOH, KOH, alkoxides over 5 mol %), and then converting the resulting, highly alkaline reaction mixtures, which consist of a mixture of oxethylated alcohols and alkoxides of different polyalkylene glycol ethers, into the corresponding ether carboxylic acid in a classic Williamson synthesis with sodium chloroacetate. Although this process reduces the residual content of fatty alcohol in the ether carboxylic acid without special catalysts, the formation of the by-products described above cannot be avoided.

The object was therefore to develop a process for the preparation of ether carboxylic acids through which the content of undesired by-products, such as sodium chloride and glycolic acid, can be reduced.

Surprisingly, it has been found that the ether carboxylic acids obtained by a washing process with sulfate solution not only have a smaller proportion of by-products, but, in particular, also have a lower setting point than ether carboxylic acids prepared by conventional methods. Furthermore, the investigations revealed that these ether carboxylic acids also have an unexpectedly low electrolyte content, which can be verified directly by conductivity measurements, and which clearly determines the setting point behavior.

The invention therefore provides a process for the preparation of compounds of the formula (1)

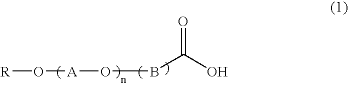

in which

A is C₂- to C₄-alkylene,

B is C₁- to C₄-alkylene

n is a number from 1 to 100, and

R is C₁- to C₃₀-alkyl, C₂- to C₃₀-alkenyl, or C₆- to C₃₀-aryl,

by alkylating a basic mixture of oxethylated alcohols of the formula

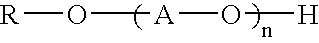

and alkoxides thereof with a C₂- to C₅-chlorocarboxylic acid, and purifying the basic intermediate obtained in this way, following acidification, by washing with aqueous sulfate solution until the ether carboxylic acid obtained in this way has a conductivity of <1000 μS/cm.

The invention further provides for the use of sulfuric acid for the acidification of the resulting basic intermediate and thus the generation of the sulfate solution required for the washing in situ.

The invention further provides for the use of the compounds of the formula 1 prepared by this process and/or salts thereof of the formula 2

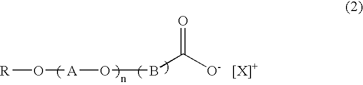

in which A, n, B and R have the meanings given above, and X is a cation, as emulsifiers, in particular as metal-working compositions, in cosmetic formulations, and as detergents in washing compositions. Preference is given to the use as metal-working compositions.

A is preferably propylene or ethylene, in particular ethylene. In a further preferred embodiment of the invention, the group —(A—O)ₙ— is a mixed alkoxy group which can contain ethylene, propylene and butylene radicals. If it is a mixed alkoxy group, then the ratio of the groups derived from the ethylene oxide to the groups derived from propylene oxide or butylene oxide is preferably between 10:1 and 1:1.

n is preferably a number between 2 and 70, in particular 3 to 50.

B is preferably a straight-chain alkylene group, in particular methylene. B can also be a branched alkylene group having 3 or 4 carbon atoms.

In a preferred embodiment, R is a C₈-C₂₄, in particular a C₁₂-C₁₈-alkyl or alkenyl radical. If R is an aromatic radical, then a phenyl radical with alkyl substitution between 4 and 12 carbon atoms is preferred.

In a preferred embodiment, X can be hydrogen ions. In a further preferred embodiment, X is alkali metal or alkaline earth metal ions, in particular lithium, sodium, potassium, magnesium or calcium.

In a further preferred embodiment, the cations used are ammonium ions of the formula NR¹R²R³R⁴, where R¹, R², R³ and R⁴, independently of one another, may be H, C₁- to C₂₂-alkyl, C₆- to C₁₈-aryl, C₇- to C₂₂-alkylaryl and/or C₁- to C₂₂-alkenyl. The radicals R¹, R², R³ and R⁴ can contain heteroatoms such as N, P, O, S. The ammonium radicals can be monoalkylammonium, dialkylammonium, trialkylammonium or tetraalkylammonium radicals in which the alkyl substituents, independently of one another, may be occupied by up to 3 hydroxyl groups. Preferably, X is ammonium radicals which carry one, two, three or four C₂- to C₁₀-alkyl radicals. In a further preferred embodiment, one, two or three of the radicals R¹ to R⁴ may be alkoxylated.

Suitable amines for the preparation of ammonium cations X are monoamines with primary or secondary amino function, such as methylamine, ethylamine, butylamine, laurylamine, coconut fatty amine, stearylamine, dimethylamine, diethylamine, dibutylamine, but also di- and polyamines, such as, for example, 3-dimethylaminopropylamine, 3-diethylaminopropylamine, 3-morpholinopropyl-amine, diethylenetriamine, triethylenetetramine or tetraethylenepentamine.

Suitable aminoalcohols for the preparation of ammonium cations X are, for example, N,N-dimethylaminoethanol, N,N-diethylaminoethanol, N,N-dibutylaminoethanol, 3-dimethylaminopropanol, N-hydroxyethylmorpholine, monoethanolamine, diethanolamine, triethanolamine, 3-aminopropanol, isopropanolamine, 2-(2-aminoethoxy)ethanol and cyclohexylamino-N,N-diethanol.

Suitable base fatty alcohols for the process described here are linear or branched, saturated or unsaturated fatty alcohols having 1-30 carbon atoms, and alkylphenols having a C₁-C₂₀-alkyl radical, preference being given to C₆-C₂₂-fatty alcohols.

According to the prior art, these can be reacted with alkylene oxides, e.g. ethylene oxide, propylene oxide, butylene oxide or mixtures of different such alkylene oxides, preference being given to, ethylene oxide or mixtures of ethylene oxide and propylene oxide. Based on fatty alcohol, 1-30 mol of alkylene oxide are supplied, preferably 1-12 mol. The reaction temperatures here are about 80-160° C.

In the subsequent reaction step, the alkoxide/alcohol oxethylate mixture is reacted with a chlorocarboxylic acid derivative and a base, preferably dry sodium chloroacetate and sodium hydroxide. This can be carried out by reacting the oxethylate/alkoxide mixture with 100-150 mol % of sodium chloroacetate at 30-100° C. and, at the same time or subsequently, adding solid sodium hydroxide or potassium hydroxide so that the sum of the base present in the oxethylate/alkoxide mixture and the amount of base additionally added corresponds to the amount of sodium chloroacetate.

Following the alkylation reaction, the intermediate solution of the ether carboxylic acid alkali metal salt can be acidified to pH<3 by acidification to pH<3 with any desired acid. The free ether carboxylic acid obtained in this way is, after the aqueous phase has been separated off, purified repeatedly by washing with a sulfate solution.

The acidification is preferably carried out with sulfuric acid since in this way alkali metal sulfate solution is already generated in situ, with which the first washing step can be carried out. The acidification can also take place with hydrochloric acid.

The sulfate solution used is preferably saturated sodium sulfate solution.

Isolation and washing process of the ether carboxylic acid takes place by uniform thorough mixing and subsequent phase separation above the cloud point.

As the following examples show, using the process disclosed here it is possible to prepare ether carboxylic acids with a low setting point and a low electrolyte content.

### EXAMPLES

### Preparation Process

### Example 1 (Oleyl Alcohol+10 EO−ECA, Acidification with H₂SO₄)

412 g (0.565 mol) of oleyl alcohol+10 EO (e.g. Genapol O 100) were introduced into a 2 l stirred apparatus with nitrogen blanketing and heated to 40° C. Then, with thorough stirring, 92.0 g (0.79 mol) of sodium chloroacetate were introduced and the reaction mixture was heated to 50° C. Then, a total of 35.0 g (0.88 mol) of sodium hydroxide microprills were added in portions such that the internal temperature does not exceed 55° C. After each addition, the mixture was stirred for 30 min, and after the last addition for 2 h, at 70° C. The reaction mixture was then heated to 90° C. and then warm sulfuric acid (15-20% strength) was allowed to run in until a pH of <3 was reached. The reaction mixture was then uniformly mixed, heated to about 100° C. and transferred to a heatable separation vessel with stirrer and bottom valve. Phase separation was carried out without stirring at a temperature of about 100-110° C., where, after a separation time of about 5 h, 552 g of aqueous lower phase and 448 g of product in the form of a pale yellow liquid were obtained.

### Example 2 (Oleyl Alcohol+10 EO−ECA, Acidification with H₂SO₄ and Washing with Sodium Sulfate Solution)

Preparation of the oleyl alcohol+10 EO−ECA was carried out in accordance with example 1. After the aqueous lower phase had been separated off, 90 g of a 25-28% strength sodium sulfate solution in water were added and the mixture was mixed vigorously for 30 min at about 100° C. Phase separation was again carried out after 2 h without stirring at a temperature of about 100-110° C., the washing phase being drawn off through the bottom valve. The washing is repeated at least 3 times. 427 g of product in the form of a pale yellow liquid were obtained.

### Example 3 (Oleyl Alcohol+10 EO−ECA, Acidification with HCl and Washing with Sodium Sulfate Solution)

412 g (0.565 mol) of oleyl alcohol+10 EO (e.g. Genapol O 100) were introduced into a 2 l stirred apparatus with nitrogen blanketing and heated to 40° C. Then, with thorough stirring, 92.0 g (0.79 mol) of sodium chloroacetate were introduced and the reaction mixture was heated to 50° C. Then, a total of 35.0 g (0.88 mol) of sodium hydroxide microprills were added in portions such that the internal temperature does not exceed 55° C. After each addition, the mixture was stirred for 30 min, and after the last addition for 2 h, at 70° C. The reaction mixture was then heated to 90° C. and then warm hydrochloric acid (35% strength) was allowed to run in until a pH of <3 was reached. The reaction mixture was then uniformly mixed, heated to about 100° C. and transferred to a heatable separation vessel with stirrer and bottom valve. Phase separation was carried out after a separation time of about 5 h without stirring at a temperature of about 100-110° C. After the aqueous lower phase had been separated off, 90 g of a 25-28% strength sodium sulfate solution in water were added and the mixture was mixed vigorously for 30 min at about 100° C. Phase separation was carried out after 2 h again without stirring at a temperature of about 100-110° C., the washing phase being drawn off through the bottom valve. The washing is repeated at least 3 times. After the last washing step, 440 g of product were obtained in the form of pale yellow liquid.

TABLE 1
Characteristics of the ether carboxylic acids (AN = acid number)

| | | | | | AN | |
|---|---|---|---|---|---|---|
| | | | | | after | |
| | NaCl | Sulfate | | AN | storage | Setting |
| | content | content | Conductivity | [mg | [mg | point |
| Example | [%] | [%] | [μS/cm] | KOH/g] | KOH/g] | [° C.] |
| Compara- | 0.64 | 0 | 1503 | 70.2 | 61.1 | 28 |
| tive | | | | | | |
| 1 | 0.36 | 0.63 | 2216 | 74.9 | 74.6 | 22 |
| 2 | 0.046 | 0.036 | 751 | 76.6 | 76.0 | 12 |
| 3 | 0.081 | 0.042 | 805 | 67.7 | 67.5 | 12 |

The comparison used was the commercially available ether carboxylic acid Emulsogen® COL 100. This is essentially an ether carboxylic acid of composition oleyl-O-(EO)₁₀-CH₂—COOH which has been prepared by a process of the prior art.

As can be seen from table 1, the ether carboxylic acids prepared by the process disclosed here are characterized by low electrolyte content, which manifests itself in a low conductivity. Furthermore, the low electrolyte content leads to the secondary effect that the ether carboxylic acids have a significantly changed setting point behavior, which simplifies the use of the products at low temperatures by the consumer.

A further advantage of the ether carboxylic acids prepared by this process arises from the relatively long storage stability, which is documented by the unchanged acid numbers following storage.

B) Use of the Compounds According to the Invention as Corrosion Inhibitor for Water-Miscible Cutting Fluids, Cleaning Liquids, and for Surface Treatments.

The corrosion protection test was carried out in accordance with DIN Standard 51360, part 2 (filter paper test) and is used to assess the corrosion of iron metal. A measure of the corrosion is the type and number of corrosion marks on a round filter which form as a result of the action of a cutting fluid (CF) mixed with water on standardized gray iron turnings (turning size: 3 to 6 mm²). The assessment is made by means of a visual test and grading of the degree of corrosion (1 to 4) according to a comparison table. The comparison used was likewise the commercially obtainable ether carboxylic acid Emulsogen® COL 100.

The products to be tested were adjusted to pH 9.0 for the investigations relating to corrosion protection using triethanolamine (TEA) to form the corresponding ammonium salt.

TABLE 2
Corrosion protection test in accordance with DIN (filter paper test),
data in corrosion grades 1 to 4 in accordance with the comparison table
in DIN Standard 51360, part 2 (filter paper test), concentrations in % by
weight

| | | Concentration of the ECA | | |
|---|---|---|---|---|
| Example | ECA | 3% | 4% | 5% |
| 4 | Comparison | 4 | 2 | 1 |
| 5 | 1 | 4 | 2 | 1 |
| 6 | 2 | 4 | 1 | 0-1 |
| 7 | 3 | 4 | 1 | 1 |

As table 2 shows, the low electrolyte content leads not only to low setting points, but also to improved corrosion protection behavior of the ether carboxylic acids according to the invention.

1. A process for the preparation of compounds of the formula (1) 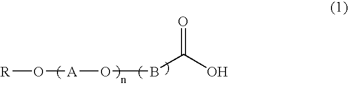
in which
A is C₂- to C₄-alkylene,
B is C₁- to C₄-alkylene
n is a number from 1 to 100, and
R is C₁- to C₃₀-alkyl, C₂- to C₃₀-alkenyl, or C₆- to C₃₀-aryl,
by alkylating a basic mixture of oxethylated alcohols of the formula 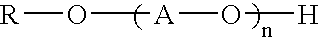
and alkoxides thereof with a C₂- to C₅-chlorocarboxylic acid, and purifying the basic intermediate obtained in this way, following acidification, by washing with aqueous sulfate solution until the ether carboxylic acid obtained in this way has a conductivity of <1000 μS/cm.

2. The process as claimed in claim 1, in which A is propylene or ethylene.

3. The process as claimed in claim 1 and/or 2, in which n is a number between 2 and 70.

4. The process as claimed in one or more of claims 1 to 3, in which B is a methylene group.

5. The process as claimed in one or more of claims 1 to 4, in which R is a C₈- to C₂₄-alkyl or alkenyl radical.

6. The process as claimed in one or more of claims 1 to 5, in which sulfuric acid is used for the acidification.

7. The use of the compounds of the formula 1 prepared by the process as claimed in one or more of claims 1 to 6, and/or salts thereof of the formula 2 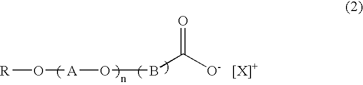
in which X is a cation, as emulsifier with anticorrosive properties.

8. The use as claimed in claim 7 in metal-working compositions, cosmetic formulations or washing compositions.
